(19) United States
(12) Patent Application Publication
(10) Pub. No.: US 2023/0375200 A1
(43) Pub. Date: Nov. 23, 2023

(54) COMPOSITE AIR STERILIZATION PURIFIER

(71) Applicant: NIDS CO., LTD., Seongnam-si, Gyeonggi-do (KR)

(72) Inventor: Hak In HWANG, Seoul (KR)

(73) Assignee: NIDS CO., LTD., Seongnam-si, Gyeonggi-do (KR)

(21) Appl. No.: 18/029,088

(22) PCT Filed: Sep. 15, 2021
(86) PCT No.: PCT/KR2021/012538

§ 371 Date: Mar. 28, 2023

(30) Foreign Application Priority Data
Sep. 29, 2020 (KR) 10-2020-0127243

(51) Int. Cl. F24F 8/00; F24F 8/108; F24F 8/192; F24F 8/22; F24F 8/167; F24F 8/158; A61L 9/20; A61L 9/22

(52) U.S. Cl. CPC F24F 8/00; F24F 8/108; F24F 8/194; F24F 8/22; F24F 8/167; F24F 8/158; A61L 9/205; A61L 9/22; A61L 2209/14; A61L 2209/11

(57) ABSTRACT

A composite air sterilization purifier includes: a particle separation part for separating, from bacteria and viruses in the air, dust in the air suctioned through the intake port; an electrostatic precipitation part for collecting dust, bacteria and viruses in the air having passed through the particle separation part; an ultraviolet sterilization part, which emits UVC at the electrostatic precipitation part to remove the bacteria and viruses collected in the electrostatic precipitation part; a photocatalyst filter for sterilizing the air having passed through the ultraviolet sterilization part; a plasma sterilization part, which increases the density of the bacteria and the viruses in the air having passed through the photocatalyst filter, so as to intensively remove the bacteria and the viruses; and an active species filter for absorbing harmful gas, ozone, and residual active species in the air having passed through the plasma sterilization part.

### TECHNICAL FIELD

The present invention relates to an air purifier, and more particularly to a composite air sterilization purifier capable of removing ultrafine dust included in air and at the same time effectively eradicating bacteria and nanoscopic viruses to purify the air.

### BACKGROUND ART

It is publicly known that infection spreads through splash and re-scattering of viruses in air and generation of high-density fine dust is related to spread of viruses, and therefore it becomes important to remove bioaerosols in air and to remove nanoscopic microorganisms, such as viruses.

HEPA filtration and electrostatic dust collection are typical technologies capable of effectively capturing harmful microorganisms in air, such as viruses.

Here, an air purifier including an antibacterial filter formed by coating a filter with an antibacterial material in order to eradicate harmful microorganisms has an antibacterial function of generating a cluster of ions at the front end of the filter such that the ions react with microorganisms attached to the filter to eradicate the microorganisms and a sterilization function of allowing microorganisms to pass through plasma to eradicate the microorganisms.

However, the conventional antibacterial filter has a problem in that large-particle polluted matter easily accumulates on the antibacterial filter, whereby it is not possible to effectively eradicate harmful nanoscopic microorganisms.

In other words, an air purifier that removes fine dust using a HEPA filter has a problem in that a high fluid pressure loss occurs due to the HEPA filter, whereby the clean air delivery rate (CADR) is reduced, and therefore it is not possible for the air purifier to effectively and instantaneously sterilize air polluted by viruses.

It is known that plasma is effective in eradicating bacteria and viruses in air; however, there is a problem in that an active contact plasma zone capable of eradicating the bacteria and the viruses is narrow and radicals cannot spread far by a fan, whereby effective reaction does not occur.

In addition, there is a problem in that air current cannot be effectively forwarded to a high-density plasma zone and if operating voltage is increased to high voltage in order to generate a large number of plasma radicals, an unpleasant smell of ozone occurs.

That is, when the HEPA filter and the plasma device are disposed in a closed flow channel structure, both have the above problems.

Meanwhile, various kinds of air sterilization purifiers having new technology applied thereto have been developed in recent years. For example, an air sterilization deodorizer using high-efficiency plasma, UV, and catalyst, which is air sterilization technology, is disclosed in Korean Patent Application Publication No. 10-2020-0079911 (2020.07.06).

In the above prior art, technology of capturing and sterilizing dust in air and discharging the air is disclosed; however, various constructions configured to effectively sterilize viruses are not provided, and a method capable of solving a problem in that the function of a photocatalyst is reduced due to introduction of moisture is not implemented.

### DISCLOSURE

### Technical Problem

The present invention has been made in view of the above problems, and it is an object of the present invention to provide a composite air sterilization purifier capable of, in purifying particulate microorganisms, maximizing efficiency in capture of nanoparticles including harmful microorganisms through the use of an electrostatic dust collection type filter having a structure that does not disturb the flow of air while having a large area, such as a honeycomb structure, directly and continuously radiating ultraviolet light to harmful microorganisms captured in an electrostatic dust collection unit to primarily collectively sterilize the harmful microorganisms, capturing droplet moisture from microorganisms that pass through the electrostatic dust collection unit in a state of being stuck on large particles, such as saliva droplets, through a porous dehumidification layer of a photocatalyst filter, secondarily sterilizing the microorganisms as the result of a photocatalyst reaction effect, and tertiarily directly sterilizing nanoparticle microorganisms that have passed through the dehumidification layer through a venturi structure in which the nanoparticle microorganisms are dispersed into a plurality of plasma generators and the flow of air converges into a high-density plasma zone.

It is another object of the present invention to provide a composite air sterilization purifier capable of, in order to improve efficiency in sterilizing bacteria and viruses separated through a particle separation unit, introducing the bacteria and the viruses into a plasma zone having a narrow flow channel in a plasma sterilization unit such that the density of the bacteria and viruses is increased, thereby intensively sterilizing the bacteria and the viruses that pass through the plasma zone.

### Technical Solution

In order to accomplish the above objects, the present invention provides a composite air sterilization purifier configured to forcibly suction external air through an intake port in order to sterilize the air and to discharge the purified air through an exhaust port, the composite air sterilization purifier including a particle separation unit configured to separate dust included in the air suctioned through the intake port and bacteria and viruses included in the air from each other, an electrostatic dust collection unit configured to capture the dust, the bacteria, and the viruses included in the air that has passed through the particle separation unit, an ultraviolet sterilization unit configured to radiate UVC to the electrostatic dust collection unit in order to sterilize the bacteria and the viruses captured in the electrostatic dust collection unit, a photocatalyst filter configured to sterilize the air that has passed through the ultraviolet sterilization unit, a plasma sterilization unit configured to increase the density of the bacteria and the viruses included in air that has passed through the photocatalyst filter and to intensively sterilize the bacteria and the viruses, and an active species filter configured to absorb harmful gas, ozone, and residual active species included in the air that has passed through the plasma sterilization unit.

The particle separation unit may separate the dust and the bacteria and the viruses having a smaller particle size than the dust from each other based on particle size.

The electrostatic dust collection unit may be configured such that a HEPA filter and an electric dust collection filter are horizontally disposed in parallel and such that the HEPA filter and the electric dust collection filter are vertically mounted in slots of the electrostatic dust collection unit so as to be detachable therefrom.

The air including the dust that has passed through the particle separation unit may be introduced into the HEPA filter, and the air including the bacteria and the viruses that have passed through the particle separation unit may be introduced into the electric dust collection filter.

The electric dust collection filter may include a dust collection electrode unit configured to have a structure in which dust collection electrodes are alternately disposed and a switching unit configured to select the polarity of each of the dust collection electrodes.

The ultraviolet sterilization unit may radiate UVC to the photocatalyst filter to sterilize bacteria and viruses adsorbed on the photocatalyst filter.

The composite air sterilization purifier may further include a plasma sterilization unit provided between the electrostatic dust collection unit and the ultraviolet sterilization unit, wherein the plasma sterilization unit may be configured to sterilize the bacteria and the viruses included in the air that has passed through the electric dust collection filter.

The plasma sterilization unit may be provided with an air inlet configured to allow air to be introduced therethrough and an air outlet configured to allow the air introduced through the air inlet to be discharged therethrough, a plasma zone may be formed between the air inlet and the air outlet, the plasma zone being configured to allow air to pass between plasma electrodes spaced apart from each other so as to correspond to each other therein, and the plasma zone may be formed in a venturi structure having a predetermined radius of curvature in section.

Each of the plasma electrodes may be disposed so as to be inclined at a predetermined angle toward the air inlet such that the end of each of the plasma electrodes faces the air inlet.

The composite air sterilization purifier may further include an inlet air quality sensor provided in the intake port, the inlet air quality sensor being configured to sense at least one of dust, harmful gas, bacteria, and viruses included in the air suctioned through the intake port, and a controller configured to control the electrostatic dust collection unit, the ultraviolet sterilization unit, and the plasma sterilization unit according to a sensing signal from the inlet air quality sensor.

The composite air sterilization purifier may further include a suction fan provided in the exhaust port and a fan motor configured to provide torque to the suction fan, wherein the fan motor may be controlled by the controller such that the amount of air that is suctioned through the intake port is adjusted based on the degree of air pollution sensed by the inlet air quality sensor.

### Advantageous Effects

The present invention has an effect in that any kind of charged microorganisms or microorganisms charged with electricity may be collected through a dust collection electrode plate configured such that a plurality of positive (+) electrodes and a plurality of negative (−) electrodes configured to electrically capture nanoscopic microorganism particles included in air are alternately disposed, whereby it is possible to obtain a very efficient microorganism collection effect, and microorganisms captured by an electrode plate are primarily sterilized through ultraviolet light and microorganisms introduced into a plasma zone are secondarily sterilized, whereby it is possible to eradicate the microorganisms.

In addition, the present invention has an effect in that, in order to improve efficiency in sterilizing bacteria and viruses separated through a particle separation unit, the bacteria and the viruses are introduced into a plasma zone having a narrow flow channel in a plasma sterilization unit such that the density of the bacteria and viruses is increased, whereby it is possible to intensively sterilize the bacteria and the viruses that pass through the plasma zone.

### DESCRIPTION OF DRAWINGS

FIG. 1 is a view schematically showing the construction of a composite air sterilization purifier according to an embodiment of the present invention.

FIG. 2 is a partial sectional view showing the composite air sterilization purifier according to the embodiment of the present invention.

FIG. 3 is a view schematically showing an electric dust collection filter of the composite air sterilization purifier according to the embodiment of the present invention.

FIG. 4 is a partial sectional view showing the composite air sterilization purifier according to the embodiment of the present invention.

FIGS. 5 and 6 are views schematically showing a plasma sterilization unit of the composite air sterilization purifier according to the embodiment of the present invention.

### DESCRIPTION OF REFERENCE SYMBOLS

10: Intake port 11: Inlet air quality sensor
20: Exhaust port 21: Outlet air quality sensor
22: Suction fan 23: Fan motor
100: Particle separation unit 200: Electrostatic dust collection unit
210: HEPA filter 220: Electric dust collection filter
221: Dust collection electrode unit 221a, 221b: Dust collection electrodes
222: Switching unit 300: Ultraviolet sterilization unit
310: UVC light source 400: Photocatalyst filter
500, 700: Plasma sterilization units 500a, 700a: Plasma electrodes
510, 710: Air inlets 520, 720: Plasma zones
530, 730: Air outlets 600: Active species filter
800: Controller

### BEST MODE

Hereinafter, a preferred embodiment of a composite air sterilization purifier according to the present invention will be described in detail with reference to the accompanying drawings.

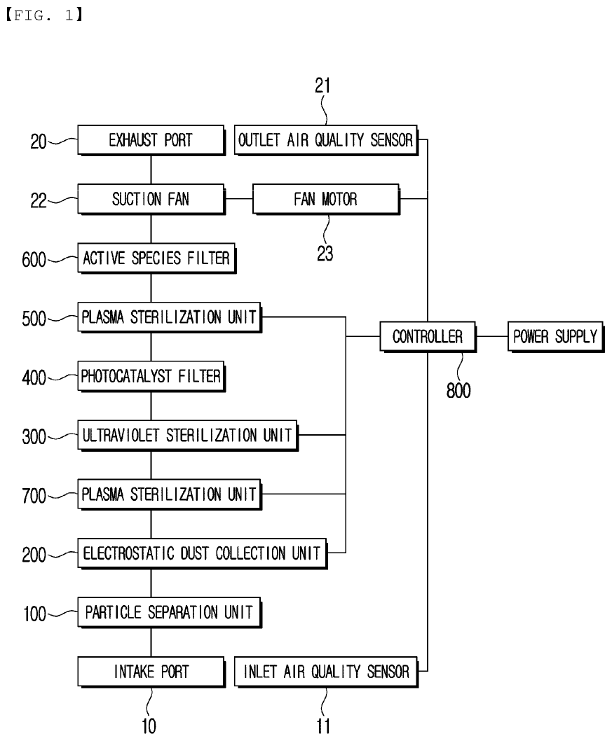

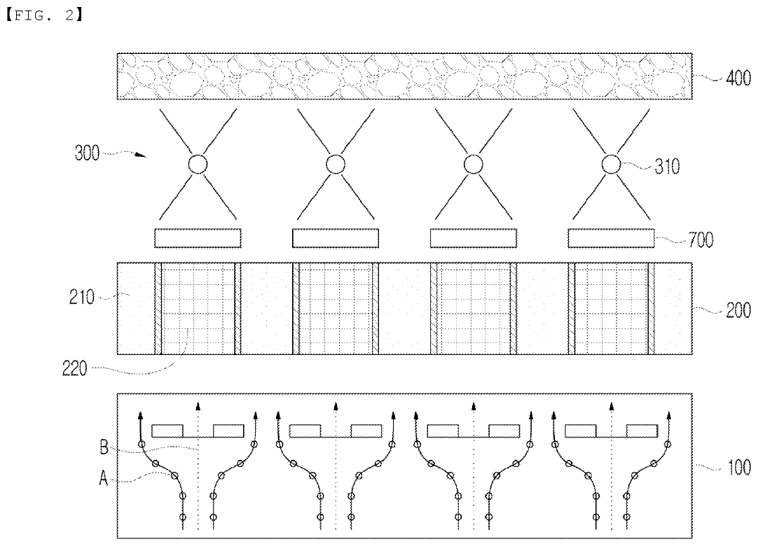

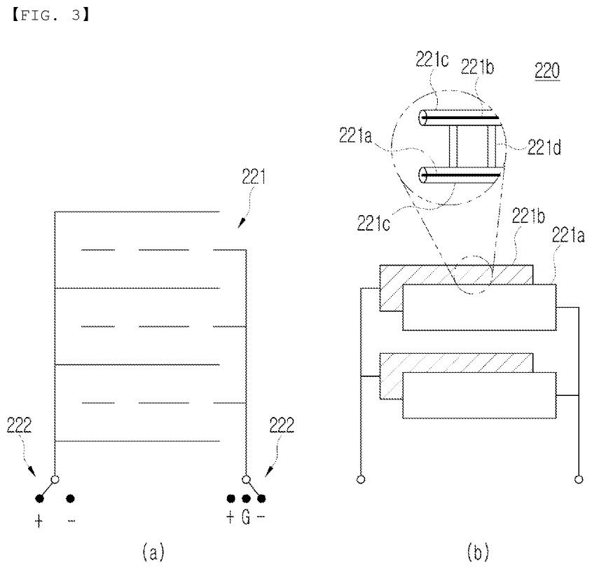

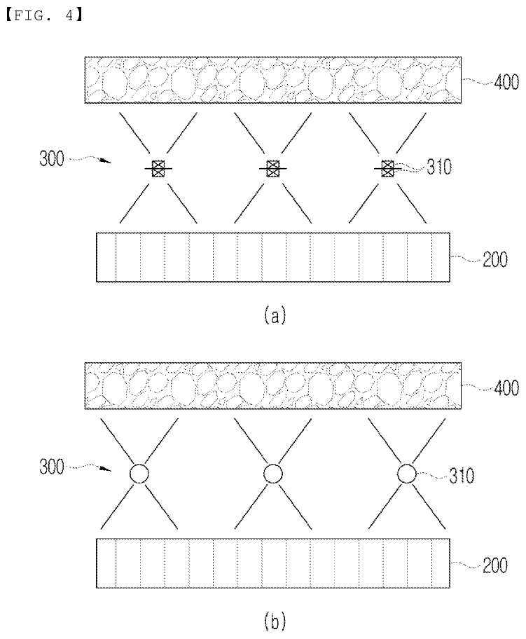

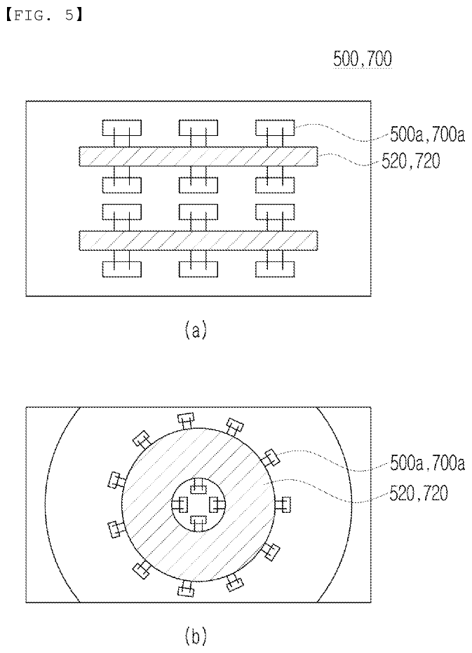

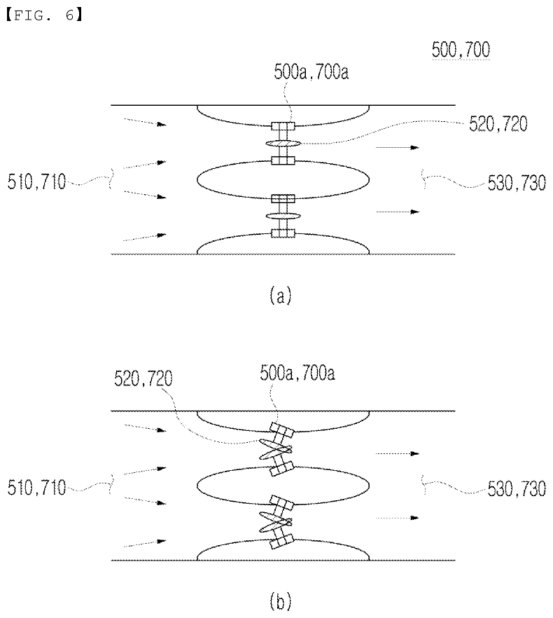

FIG. 1 is a view schematically showing the construction of a composite air sterilization purifier according to an embodiment of the present invention, FIG. 2 is a partial sectional view showing the composite air sterilization purifier according to the embodiment of the present invention, FIG. 3 is a view schematically showing an electric dust collection filter of the composite air sterilization purifier according to the embodiment of the present invention, FIG. 4 is a partial sectional view showing the composite air sterilization purifier according to the embodiment of the present invention, and FIGS. 5 and 6 are views schematically showing a plasma sterilization unit of the composite air sterilization purifier according to the embodiment of the present invention.

Referring to FIGS. 1 to 6, the composite air sterilization purifier according to the preferred embodiment of the present invention includes a particle separation unit 100, an electrostatic dust collection unit 200, an ultraviolet sterilization unit 300, a photocatalyst filter 400, a plasma sterilization unit 500, and an active species filter 600, which will be described hereinafter in detail.

The composite air sterilization purifier according to the present invention is a composite air sterilization purifier that has a multistage structure in which function-based filters and function-based sterilization units are assembled with each other while being fastened to each other in a stacked state and that forcibly suctions external indoor air through an intake port 10 at the bottom of the composite air sterilization purifier to sterilize the air and discharges the purified air to the outside through an exhaust port 20.

The composite air sterilization purifier according to the present invention further includes an inlet air quality sensor 11 provided in the intake port 10 to sense at least one of dust, harmful gas, bacteria, and viruses included in the air suctioned through the intake port 10.

In addition, the composite air sterilization purifier according to the present invention further includes a controller 800 configured to control the electrostatic dust collection unit 200, the ultraviolet sterilization unit 300, and the plasma sterilization unit 500 according to a sensing signal from the inlet air quality sensor 11.

Furthermore, the composite air sterilization purifier according to the present invention further includes a suction fan 22 provided in the exhaust port 20 and a fan motor 23 configured to provide torque to the suction fan 22, wherein the fan motor 23 is controlled by the controller 800 such that the amount of air that is suctioned through the intake port 10 is adjusted based on the degree of air pollution sensed by the inlet air quality sensor 11.

Here, the flow rate in the intake port 10, i.e. the intake amount of air, is determined through control of the speed of the fan motor 23 according to a command of the controller 800 based on the signal from the inlet air quality sensor 11.

That is, the intake amount of air may be adjusted based on the degree of indoor air pollution sensed by the inlet air quality sensor 11. Alternatively, the intake amount of air may be adjusted according to an external sensor signal or a signal received from a control center in a wired or wireless manner.

Meanwhile, in the composite air sterilization purifier according to the present invention, a flow channel may be formed so as to extend from the intake port 10 to the exhaust port 20 via the sterilization units and the filters.

The flow channel is formed in a stacked state such that the electrostatic dust collection unit 200, the ultraviolet sterilization unit 300, the photocatalyst filter 400, the plasma sterilization unit 500, the active species filter 600, and the suction fan 22 are sequentially connected to each other or are connected to each other in a staggered state based on function.

The particle separation unit 100 is a separation device that separates dust particles A and bacteria and virus particles B from each other based on size and sends the separated particles to sections each having an effective capture mechanism.

In other words, the particle separation unit 100 serves to separate dust A included in the air suctioned through the intake port 10 and bacteria and viruses B included in the air from each other.

That is, bacteria and viruses B having a smaller particle size than dust A and dust A having a larger particle size than bacteria and viruses are separated from each other based on size through the particle separation unit 100.

In other words, dust A particles each having a size of about 1 to 20 μm and bacteria and virus particles B each having a size of about 0.1 to 0.5 μm are separated from each other through the particle separation unit 100.

As described above, the bacteria and viruses are separated from the dust through the particle separation unit 100. When the separated bacteria and viruses are introduced into plasma sterilization units 500 and 700, a description of which will follow, therefore, the density of the bacteria and viruses is increased in plasma zones 520 and 720 each having a narrow flow channel formed therein, whereby the bacteria and the viruses that pass through the plasma zones 520 and 720 are intensively sterilized, and therefore sterilization efficiency is improved.

The particle separation unit 100 according to the present invention, which is generally used in the particle measurement (instrumentation) field, is known from Korean Registered Patent Publication No. 10-1290558, No. 10-1338349, No. 10-0942364, No. 10-1149356, and No. 10-1622342, and therefore a detailed description thereof will be omitted.

The electrostatic dust collection unit 200 serves to capture and remove dust, bacteria, and viruses included in the air that has passed through the particle separation unit 100.

In the electrostatic dust collection unit 200, a HEPA filter 210 configured to collect ultrafine dust (PM 1.0 or less) and an electric dust collection filter 220 are horizontally disposed in parallel.

The HEPA filter 210 and the electric dust collection filter 220 are vertically mounted in slots of the electrostatic dust collection unit 200 so as to be detachable from the electrostatic dust collection unit.

Here, the air including the dust that has passed through the particle separation unit 100 is introduced into the HEPA filter 210, and the air including the bacteria and the viruses that have passed through the particle separation unit 100 is introduced into the electric dust collection filter 220.

Meanwhile, the electric dust collection filter 220 includes a dust collection electrode unit 221 configured to have a structure in which dust collection electrodes 221a and 221b are alternately disposed and a switching unit 222 configured to select the polarity of each of the dust collection electrodes 221a and 221b.

In other words, the dust collection electrodes 221a and 221b of the electric dust collection filter 220 are alternately disposed in sequence to generate an electric field.

The dust collection electrodes 221a and 221b are arranged long in a flow direction of air so as to provide a three-dimensional honeycomb structure. Since the electric dust collection filter 220 can be easily detached from the electrostatic dust collection unit 200, the electric dust collection filter can be washed using water. In addition, the electric dust collection filter can efficiently collect the bacteria and viruses in an electrostatic manner even when the flow rate is high.

In the dust collection electrode unit 221, which has a large high-pressure area, of the electric dust collection filter 220, a plurality of positive (+) dust collection electrodes 221a and a plurality of negative (−) dust collection electrodes 221b are alternately disposed.

In general, polluted particles in air, which are positively (+) charged, are collected by the negative (−) dust collection electrodes 221b, whereas nanoparticles, which are negatively (−) charged, are collected by the positive (+) dust collection electrodes 221a.

The switching unit 222 may change the polarity of each of the dust collection electrodes 221a and 221b according to a signal from the controller 800 such that the dust collection electrodes 221a and 221b can alternately collect charged particles or can be charged with electricity.

In other words, the dust collection electrode unit 221 is configured to have a structure in which the positive (+) dust collection electrodes 221a and the negative (−) dust collection electrodes 221b are alternately disposed in a line in the state in which each of the positive (+) dust collection electrodes and a corresponding one of the negative (−) dust collection electrodes are spaced apart from each other by a predetermined distance due to an insulative support 221d interposed therebetween.

Here, each of the dust collection electrodes 221a and 221b has a metal ribbon or wire mesh structure in which the length of each of the dust collection electrodes 221a and 221b is sufficient to efficiently collect charged particles even when the flow rate of air is increased.

For neutrally charged particles, the switching unit 222 may be adjusted to selectively switch between polarities (+, −, and G) of the power supply. After charged with the selected polarity, the particles may be effectively collected by a dust collection electrode having polarity opposite the polarity of the particles installed at the rear.

Dust collection efficiency of the electric dust collection filter 220 may be measured by an outlet air quality sensor 21 provided in the exhaust port 20, whereby dust collection performance of the electrostatic dust collection unit 200 may be measured in real time, and the polarity of each of the dust collection electrodes 221a and 221b may be selectively controlled, whereby the dust collection electrodes may be efficiently controlled based on the charged state of particles.

Meanwhile, negatively (−) charged particles may be collected in the state in which only positive (+) voltage is always applied, and positively (+) charged particles may be collected in the state in which only negative (−) voltage is always applied.

Alternatively, an electric field may be applied to neutral particles to polarize the neutral particles, and the polarized neutral particles may be collected by dust collection electrodes installed at the rear. This is adjustable depending on the type of sensed particles.

In other words, the electric dust collection filter 220 has a three-dimensional structure including electrodes that are alternately disposed and are configured to capture nanoscopic particles, such as bacteria or viruses, among particulate matter included in air, using an electrical method.

When particulate matter passes through the electric dust collection filter 220, the particulate matter is collected by a dust collection electrode having polarity opposite the polarity of charged particulate matter, and uncharged particulate matter may be charged by the dust collection electrodes during passage through the electric dust collection filter and may be collected by the next stage dust collection electrode having polarity opposite the polarity of the charged particulate matter.

Here, each of the dust collection electrodes 221a and 221b may have a metal ribbon or wire mesh structure in which each of the dust collection electrodes is coated with an insulator (dielectric film) 221c, in which a plurality of positive (+) electrodes and a plurality of negative (−) electrodes 221b are alternately disposed, and in which an insulative spacer 221d is interposed between neighboring ones of the dust collection electrodes 221a and 221b to maintain a uniform distance therebetween.

That is, in purifying particulate microorganisms, it is possible to maximize efficiency in capture of nanoparticles including harmful microorganisms through the use of an electrostatic dust collection type filter having a structure that does not disturb the flow of air while having a large area, such as a honeycomb structure.

Consequently, any kind of charged microorganisms or microorganisms charged with electricity may be collected through a dust collection electrode plate configured such that a plurality of positive (+) electrodes and a plurality of negative (−) electrodes configured to electrically capture nanoscopic microorganism particles included in air are alternately disposed, whereby it is possible to obtain a very efficient microorganism collection effect.

Meanwhile, the composite air sterilization purifier according to the present invention may further include a plasma sterilization unit 700 provided between the electrostatic dust collection unit 200 and the ultraviolet sterilization unit 300 to sterilize the bacteria and the viruses included in the air that has passed through the electric dust collection filter 220.

The ultraviolet sterilization unit 300 serves to radiate UVC to the electrostatic dust collection unit 200 in order to sterilize bacteria and viruses captured in the electrostatic dust collection unit 200.

In another embodiment, the ultraviolet sterilization unit 300 may also serve to radiate UVC to the photocatalyst filter 400 in order to sterilize bacteria and viruses adsorbed on the photocatalyst filter 400.

The ultraviolet sterilization unit 300 functions to sterilize bacteria or viruses collected in the electrostatic dust collection unit 200. In general, an LED 310 or a lamp 310 having a UVC wavelength may be used as the ultraviolet sterilization unit. The ultraviolet sterilization unit may be disposed close to the photocatalyst filter 400 provided thereabove to impart a sterilization function to the photocatalyst filter 400.

Meanwhile, the ultraviolet sterilization unit 300 may be configured to effectively radiate light to the entirety of the electrostatic dust collection unit 200 and inner walls of the dust collection electrodes 221a and 221b of the electrostatic dust collection unit 200 and at the same time to radiate ultraviolet light to the photocatalyst filter 400 provided thereabove, and the sterilization cycle of the ultraviolet sterilization unit may be controlled by the controller 800.

That is, the ultraviolet sterilization unit directly continuously radiates light, such as ultraviolet light, to the interior of the electrostatic dust collection unit 200 to collectively sterilize harmful microorganisms captured in the electrostatic dust collection unit 200.

The ultraviolet sterilization unit 300 may include a plurality of ultraviolet light sources 310 each having a surface light source structure configured to completely radiate light from the ultraviolet sterilization unit to the interior of the electrostatic dust collection unit 200, and the ultraviolet light sources 310 may directly or indirectly re-radiate light to the photocatalyst filter 400 provided thereabove.

The photocatalyst filter 400 decomposes gas included in the air that has passed through the ultraviolet sterilization unit 300 and sterilizes the air. The photocatalyst filter 400 is installed in a passageway through which polluted air other than the bacteria and viruses collected by the electrostatic dust collection unit 200 passes.

The photocatalyst filter may generate OH radicals through photocatalyst surface reaction by the ultraviolet sterilization unit 300 located thereunder, whereby the photocatalyst filter may have a sterilization function, and plasma OH radicals generated by the plasma sterilization unit 500 installed above the photocatalyst filter further activate photocatalyst reaction, whereby the sterilization function of the photocatalyst filter may be enhanced.

Droplet moisture in microorganisms is captured through a porous dehumidification layer of the photocatalyst filter 400, the microorganisms are sterilized by the effect of photocatalyst reaction, and nanoparticle microorganisms that have passed through the dehumidification layer are introduced into the plasma sterilization unit 500, in which the nanoparticle microorganisms are sterilized.

The photocatalyst filter 400 has a porous adsorption layer configured to effectively remove large liquid droplet particles, such as saliva, among particulate matter included in air, and therefore the photocatalyst filter adsorbs moisture and sterilizes re-scattered viruses.

The porous adsorption layer may be a structure made of an organic or inorganic material separated from a photocatalyst material, such as fiber or ceramic. The porous adsorption layer and the photocatalyst material may be integrated.

The plasma sterilization units 500 and 700 sterilize bacteria and viruses included in the air that has passed through the electrostatic dust collection unit 200 and/or the photocatalyst filter 400.

The plasma sterilization units 500 and 700 are provided respectively with air inlets 510 and 710, through which air is introduced, and air outlets 530 and 730, through which the air introduced through the air inlets 510 and 710 is discharged.

Plasma zones 520 and 720, in which air passes between plasma electrodes 500a and 700a spaced apart from each other, are formed respectively between the air inlets 510 and 710 and the air outlets 530 and 730.

Here, each of the plasma zones 520 and 720 may be formed in a venturi structure having a predetermined radius of curvature in section.

The plasma electrodes 500a and 700a may be disposed so as to be inclined at a predetermined angle toward the air inlets 510 and 710 such that ends of the plasma electrodes 500a and 700a face the air inlets 510 and 710, respectively.

Each of the plasma sterilization units 500 and 700 has a plasma sterilization and flow channel structure in which the plasma sterilization unit is three-dimensionally disposed in directions parallel to, perpendicular to, or inclined to the flow direction of air in order to increase contact area and contact time between the plasma zones 520 and 720 of the plasma sterilization units.

In other words, high-density plasma zones 520 and 720 are formed between the plasma electrodes 500a and 700a, which generate plasma, in the plasma sterilization units 500 and 700, and the plasma electrodes include a front plasma electrode disposed at the front end of the venturi structure having the predetermined radius of curvature in section in order to allow polluted air to efficiently flow and a rear plasma electrode disposed at the rear end of the venturi structure.

The plasma electrodes 500a and 700a are disposed at the front end and the rear end, respectively, the front plasma electrode has a function of sterilizing viruses while activating the photocatalyst, air that has passed through the front plasma electrode enters a high-density plasma zone provided at the rear thereof through the venturi structure, and the air is directly sterilized in the high-density plasma zone.

The plasma electrodes 500a and 700a may be arranged one after the other in plural in order to minimize resistance to the flow of air. Here, the plasma zones 520 and 720 may be formed between the high-voltage plasma electrodes 500a and 700a, each of which has a length of about 3 to 5 cm.

In other words, the plasma sterilization units 500 and 700 may be constituted by a front plasma electrode configured to generate OH radicals used to remove bacteria, viruses, and mold, a venturi structure configured to guide polluted air such that the polluted air flows to the high-density plasma zones 520 and 720, and a rear plasma electrode.

Here, OH radicals generated due to discharging between the high-voltage plasma electrodes 500a and 700a are generated through decomposition of moisture in air. The moisture in the air may be measured by a humidification sensor of a sensor unit, and the plasma electrodes 500a and 700a may effectively generate plasma under control of the controller 800. OH radicals (H₂O+e→OH+H) directly react with viruses or bacteria in polluted air in the high-density plasma zones 520 and 720, whereby the viruses or bacteria are efficiently sterilized. A plurality of independent plasma electrodes 500a and 700a may be disposed in order to effectively perform space sterilization.

The front plasma electrode may perform a sterilization function using only plasma OH radicals generated therefrom, and when a photocatalyst oxide (e.g. TiO₂) of the photocatalyst filter 400 disposed under the front plasma electrode, ultraviolet light, and the OH radicals react with each other, photocatalyst efficiency may be improved.

That is, the plasma sterilization function and the photocatalyst sterilization enhancement function are efficiently and compositely used, whereby sterilization efficiency is improved.

The density of the plasma OH radicals is highest between the two electrodes that generate plasma. When viruses or bacteria pass through the plasma zones 520 and 720, therefore, the viruses or the bacteria are very efficiently sterilized.

To this end, air may be guided so as to flow through the venturi structure such that the air flows to the high-density plasma zones 520 and 720 of the rear plasma electrode.

At this time, the plurality of plasma electrodes 500a and 700a may be disposed in order to disperse the flow of air and then to concentrate the air, whereby it is possible to reduce the air pressure difference due to narrowing of the space.

The active species filter 600 serves to finally absorb (adsorb) bacteria, viruses, decomposed harmful gas molecules, ozone, and residual active species included in the air that has passed through the plasma sterilization unit 500.

Here, an activated carbon granule or nonwoven type activated carbon filter that exhibits high breathability, exhibits excellent deodorization performance, and reacts with ozone to convert the ozone into CO₂, such as a conventionally known activated carbon filter, may be used as the active species filter 600.

Although the present invention has been described with reference to the preferred embodiment, the technical idea of the present invention is not limited thereto, it is obvious to a person having ordinary skill in the art to which the present invention pertains that modifications or alterations are possible without departing from the scope of the present invention defined by the appended claims, and such modifications or alterations fall within the scope of the present invention defined by the appended claims.

1. A composite air sterilization purifier configured to forcibly suction external air through an intake port in order to sterilize the air and to discharge the purified air through an exhaust port, the composite air sterilization purifier comprising:
a particle separation unit configured to separate dust included in the air suctioned through the intake port and bacteria and viruses included in the air from each other;
an electrostatic dust collection unit configured to capture the dust, the bacteria, and the viruses included in the air that has passed through the particle separation unit;
an ultraviolet sterilization unit configured to radiate UVC to the electrostatic dust collection unit in order to sterilize the bacteria and the viruses captured in the electrostatic dust collection unit;
a photocatalyst filter configured to sterilize the air that has passed through the ultraviolet sterilization unit;
a plasma sterilization unit configured to increase density of the bacteria and the viruses included in air that has passed through the photocatalyst filter and to intensively sterilize the bacteria and the viruses; and
an active species filter configured to absorb harmful gas, ozone, and residual active species included in the air that has passed through the plasma sterilization unit.

2. The composite air sterilization purifier according to claim 1, wherein the particle separation unit separates the dust and the bacteria and the viruses having a smaller particle size than the dust from each other based on particle size.

3. The composite air sterilization purifier according to claim 1, wherein the electrostatic dust collection unit is configured such that a HEPA filter and an electric dust collection filter are horizontally disposed in parallel and such that the HEPA filter and the electric dust collection filter are vertically mounted in slots of the electrostatic dust collection unit so as to be detachable therefrom.

4. The composite air sterilization purifier according to claim 3, wherein
the air comprising the dust that has passed through the particle separation unit is introduced into the HEPA filter, and
the air comprising the bacteria and the viruses that have passed through the particle separation unit is introduced into the electric dust collection filter.

5. The composite air sterilization purifier according to claim 3, wherein the electric dust collection filter comprises:
a dust collection electrode unit configured to have a structure in which dust collection electrodes are alternately disposed; and
a switching unit configured to select polarity of each of the dust collection electrodes.

6. The composite air sterilization purifier according to claim 1, wherein the ultraviolet sterilization unit radiates UVC to the photocatalyst filter to sterilize bacteria and viruses adsorbed on the photocatalyst filter.

7. The composite air sterilization purifier according to claim 4, further comprising a plasma sterilization unit provided between the electrostatic dust collection unit and the ultraviolet sterilization unit, the plasma sterilization unit being configured to sterilize the bacteria and the viruses included in the air that has passed through the electric dust collection filter.

8. The composite air sterilization purifier according to claim 7, wherein
the plasma sterilization unit is provided with an air inlet configured to allow air to be introduced therethrough and an air outlet configured to allow the air introduced through the air inlet to be discharged therethrough,
a plasma zone is formed between the air inlet and the air outlet, the plasma zone being configured to allow air to pass between plasma electrodes spaced apart from each other so as to correspond to each other therein, and
the plasma zone is formed in a venturi structure having a predetermined radius of curvature in section.

9. The composite air sterilization purifier according to claim 8, wherein each of the plasma electrodes is disposed so as to be inclined at a predetermined angle toward the air inlet such that an end of each of the plasma electrodes faces the air inlet.

10. The composite air sterilization purifier according to claim 1, further comprising:
an inlet air quality sensor provided in the intake port, the inlet air quality sensor being configured to sense at least one of dust, harmful gas, bacteria, and viruses included in the air suctioned through the intake port; and
a controller configured to control the electrostatic dust collection unit, the ultraviolet sterilization unit, and the plasma sterilization unit according to a sensing signal from the inlet air quality sensor.

11. The composite air sterilization purifier according to claim 10, further comprising:
a suction fan provided in the exhaust port; and
a fan motor configured to provide torque to the suction fan, wherein
the fan motor is controlled by the controller such that the amount of air that is suctioned through the intake port is adjusted based on the degree of air pollution sensed by the inlet air quality sensor.
